(19) United States
(12) Patent Application Publication
(10) Pub. No.: US 2009/0311782 A1
(43) Pub. Date: Dec. 17, 2009

(54) METHOD FOR PROMOTING DIFFERENTIATION OF STEM CELL INTO INSULIN PRODUCING CELL

(75) Inventors: Shih-Hwa Chiou, Taipei City (TW); Yu-Show Fu, Taipei City (TW); Larry Low-Tone Ho, Taipei City (TW); Shih-Chieh Hung, Taipei City (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: TAIPEI VETERANS GENERAL HOSPITAL, Taipei (TW)

(21) Appl. No.: 12/330,716

(22) Filed: Dec. 9, 2008

(30) Foreign Application Priority Data
Jun. 11, 2008 (TW) 097121807

(51) Int. Cl. C12N 5/08

(52) U.S. Cl. 435/366; 435/377

(57) ABSTRACT

A method for promoting a differentiation of stem cells into insulin producing cells is provided. The method includes steps of suspending the stem cells in a first culture medium, aggregating the stem cells to form a cell pellet, and culturing the cell pellet in a second culture medium to promote the differentiation of the stem cells of the cell pellet into the insulin producing cells.

### FIELD OF THE INVENTION

The present invention relates to a method for promoting a differentiation of stem cells, and more particularly to a method for promoting a differentiation of stem cells into insulin producing cells.

### BACKGROUND OF THE INVENTION

Islet transplantation is a potential treatment for Type I diabetes mellitus; however, such an approach has been limited by a shortage of transplantable pancreatic islet cells. One alternative to organ or tissue transplantation is to engraft a renewable source of insulin producing cells (IPCs). Stem cells have the potential to proliferate and differentiate into any type of cells, and thus provide cells which can be isolated and used for transplantation.

Islets, the principal source of insulin in humans, are derived from embryonic endoderm, but share some features with neurons. Moreover, brain neurons are the main source of circulating insulin in some invertebrate species, such as Drosophila. These and other findings suggest that cells with the neural potential may differentiate into IPCs. Multipotential mesenchymal stem cells (MSCs) can be isolated from the bone marrow, cultured and expanded in vitro. They can differentiate into multiple mesenchymal cell types, including cartilage, bone and adipose tissues. Under appropriate experimental conditions, such as treatment with growth factors, neurotrophic factors, or chemical products like retinoic acid or 3-isobutyl-1-methylxanthine (IBMX), they exhibit a neuronal phenotype. Methods promoting neural differentiation have been adapted to derive IPCs from embryonic stem cells (referring to the citations 1-4), but it remains unclear whether such methods will derive IPCs from human MSCs, or whether additional treatments other than neuronal cell-based differentiation are essential to generate transplantable IPCs.

Based on the above, since the obtainment of embryonic stem cells is difficult and results in moral dispute, and recent works suggest that embryonic stem cells-derived IPCs form teratoma after transplantation and lead to failure of treatment for Type I diabetes, it is necessary to provide a method for promoting differentiation of MSCs into IPCs, so as to provide another transplantable source for effective treatments for patients with Type I diabetes.

In order to overcome the drawbacks in the prior art, a method for promoting a differentiation of stem cells into IPCs is provided. The particular design in the present invention not only solves the problems described above, but also is easy to be implemented. Thus, the invention has the utility for the industry.

### SUMMARY OF THE INVENTION

The present invention aims to provide a method for promoting differentiation of stem cells into IPCs, so as to increase the differentiation rate of the stem cells for easy obtaining sufficient transplantable islet source.

In accordance with one aspect of the present invention, a method for promoting a differentiation of progenitor cells into IPCs is provided. The method includes steps of suspending the progenitor cells in a first culture medium, aggregating the progenitor cells to form a cell pellet, and culturing the cell pellet in a second culture medium to promote the differentiation of the progenitor cells of the cell pellet into the IPCs.

In accordance with another aspect of the present invention, a method for promoting a differentiation of stem cells into IPCs is provided. The method includes steps of suspending the stem cells in a first culture medium, aggregating the stem cells to form a cell pellet, and culturing the cell pellet in a second culture medium to promote the differentiation of the stem cells of the cell pellet into the IPCs.

Preferably, the stem cells are ones selected from a group consisting of embryonic stem cells, adult stem cells and a combination thereof.

Preferably, the embryonic stem cells are ones selected from a group consisting of embryonic germ cells, transformed embryonic stem cells, induced embryonic stem cells and a combination thereof, and the adult stem cells are ones selected from a group consisting of MSCs, hematopoietic stem cells, neural stem cells and a combination thereof.

Preferably, the MSCs are derived from a tissue being one selected from a group consisting of a bone marrow, a cord blood, an adipose tissue and an umbilical cord.

Preferably, the cell pellet includes 2.5×10⁵ stem cells, and is suspended in the second culture medium.

Preferably, the first culture medium is a complete culture medium including DMEM-low glucose, 10% fetal bovine serum, 100 U/mL penicillin and 10 μg/mL streptomycin.

Preferably, the cell pellet is aggregated by centrifuging the stem cells at 200-600 g for 5-15 minutes.

Preferably, the method further includes a step of preculturing the cell pellet in the first culture medium before the culturing step.

Preferably, the second culture medium contains one selected from a group consisting of a fibronectin, a laminin and a combination thereof.

Preferably, the culturing step includes further sub-steps of culturing the cell pellet for two days at a first stage, culturing the cell pellet for one day at a second stage, culturing the cell pellet for four days at a third stage, and culturing the cell pellet for three days at a fourth stage.

Preferably, the second culture medium is a complete culture medium at the first stage; the second culture medium is a first DMEM/F-12 medium containing insulin-transferrin-selenium-A (ITS-A), 25 mM glucose and 0.45 mM 3-isobutyl-1-methylxanthine (IBMX) at the second stage; the second culture medium is a second DMEM/F-12 medium containing N2 supplement, B27 supplement, 5.56 mM glucose and 10 mM nicotinamide at the third stage; and the second culture medium is a third DMEM/F-12 medium containing N2 supplement, B27 supplement, 25 mM glucose and 10 mM nicotinamide at the fourth stage.

Preferably, the IPCs are induced by a glucose to release an insulin, and the glucose has a concentration in a range of 5-25 mM.

Preferably, the release of the insulin is enhanced by an inhibitor of cAMP phosphodiesterase.

Preferably, the release of the insulin is inhibited by a blocker of Ca ion channel.

In accordance with a further aspect of the present invention, an IPC differentiated from a stem cell is provided, wherein the stem cell is aggregated into a cell pellet to be cultured so as to differentiate into the IPC.

Preferably, the stem cell is one selected from a group consisting of an embryonic stem cell, an adult stem cell and a combination thereof.

Preferably, the embryonic stem cell is one selected from a group consisting of an embryonic germ cell, a transformed embryonic stem cell and an induced embryonic stem cell transformed from an adult cell and a combination thereof, and the adult stem cell is one selected from a group consisting of a mesenchymal stem cell, a hematopoietic stem cell, a neural stem cell and a combination thereof.

Preferably, the differentiation of the stem cell is promoted by one selected from a group consisting of a fibronectin, a laminin and a combination thereof.

Preferably, the IPC is induced by a glucose to release an insulin, and the glucose has a concentration in a range of 5-25 mM.

Additional objects and advantages of the invention will be set forth in the following descriptions with reference to the accompanying drawings, in which:

### BRIEF DESCRIPTION OF THE DRAWINGS

FIG. 1 is a bar graph showing the relative mRNA expression levels of insulin and glucose transporter 2 (Glut2) of the control groups and in the preferred embodiments of the present invention;

FIG. 2A is a bar graph showing the relative mRNA expression levels of insulin and glucose transporter 2 (Glut2) in four culturing stages according a preferred embodiment of the present invention;

FIG. 2B is a bar graph showing the percentage of positive-stained cells for Ki67 protein in four culturing stages according a preferred embodiment of the present invention;

FIG. 2C is a bar graph showing the percentage of the percentage of positive-stained areas for nestin, β3-tubulin III, proinsulin and insulin proteins in four culturing stages according a preferred embodiment of the present invention;

FIG. 3A is a bar graph showing the insulin release at different glucose concentrations of a control group and a preferred embodiment of the present invention;

FIG. 3B is a bar graph showing the insulin release before and after treatment with IBMX and nifedipine of a preferred embodiment of the present invention;

FIG. 4A is a bar graph showing the relative mRNA expression levels of glucose transporter 2 (Glut2) of preferred embodiments of the present invention; and

FIG. 4B is a bar graph showing the relative mRNA expression levels of insulin of preferred embodiments of the present invention.

### DETAILED DESCRIPTION OF THE PREFERRED EMBODIMENT

The embodiments described below are merely exemplary and are not intended to limit the invention to the precise forms disclosed. Instead, the embodiments were selected for description to enable one of ordinary skill in the art to practice the invention.

In the following method for promoting a differentiation of stem cells into IPCs in the present invention, cells are cultured at 37° C. under 5% CO₂ atmosphere. There are two basic culture medium used in the present invention. One is complete culture medium (CCM), which contains DMEM-low glucose (LG) supplemented with 10% fetal bovine serum (FBS), 100 U/mL penicillin, and 10 μg/mL streptomycin; the other one is DMEM/F-12. According to the culture medium and the additional supplements used, there are five stages of culture period in each embodiment and control group.

### Embodiment I: Cell Pellet Suspension Culture

At Stage 0, undifferentiated human MSCs are suspended with CCM, and aliquots of 2.5×10⁵ cells are placed in 15 mL conical centrifuge tubes and centrifuged at 200-600 g for 5-15 minutes, and then are cultured in CCM for overnight.

At Stage I, the culture medium is replaced with CCM with the addition of 5 μg/mL fibronectin, and the cells are cultured for 2 days.

At Stage II, the cells are switched into a medium prepared from 1:1 mixture of DMEM/F-12 medium containing 25 mM glucose, Insulin-Transferrin-Selenium-A (ITS-A), 0.45 mM IBMX and 5 μg/mL fibronectin for 1 day.

At Stage III, the cells are transferred into DMEM/F-12 medium containing 5.56 mM glucose, 10 mM nicotinamide, N2 supplement, B27 supplement and 5 μg/mL fibronectin for 4 days.

At Stage IV, the cells are transferred into a medium with the same supplements at the stage III of the Embodiment I but containing 25 mM glucose for 3 days.

### Embodiment II: Cell Pellet Suspension Culture

At Stage 0, undifferentiated human MSCs are suspended with CCM, and aliquots of 2.5×10⁵ cells are placed in 15 mL conical centrifuge tubes and centrifuged at 200-600 g for 5-15 minutes, and then are cultured in CCM for overnight.

At Stage I, the culture medium is replaced with fresh CCM, and the cells are cultured for 2 days.

At Stage II, the cells are switched into a medium prepared from 1:1 mixture of DMEM/F-12 medium containing 25 mM glucose, ITS-A and 0.45 mM IBMX for 1 day.

At Stage III, the cells are transferred into DMEM/F-12 medium containing 5.56 mM glucose, 10 mM nicotinamide, N2 supplement and B27 supplement for 4 days.

At Stage IV, the cells are transferred into a medium with the same supplements at the stage III of the Embodiment II but containing 25 mM glucose for 3 days.

### Embodiment III: Cell Pellet Suspension Culture

At Stage 0, undifferentiated human MSCs are suspended with CCM, and aliquots of 2.5×10⁵ cells are placed in 15 mL conical centrifuge tubes and centrifuged at 200-600 g for 5-15 minutes, and then are cultured in CCM for overnight.

At Stage I, the culture medium is replaced with CCM with the addition of 5 μg/mL laminin, and the cells are cultured for 2 days.

At Stage II, the cells are switched into a medium prepared from 1:1 mixture of DMEM/F-12 medium containing 25 mM glucose, ITS-A, 0.45 mM IBMX and 5 μg/mL laminin for 1 day.

At Stage III, the cells are transferred into DMEM/F-12 medium containing 5.56 mM glucose, 10 mM nicotinamide, N2 supplement, B27 supplement and 5 μg/mL laminin for 4 days.

At Stage IV, the cells are transferred into a medium with the same supplements at the stage III of the Embodiment III but containing 25 mM glucose for 3 days.

### Embodiment IV: Cell Pellet Suspension Culture

At Stage 0, undifferentiated human MSCs are suspended with CCM, and aliquots of 2.5×10⁵ cells are placed in 15 mL conical centrifuge tubes and centrifuged at 200-600 g for 5-15 minutes, and then are cultured in CCM for overnight.

At Stage I, the culture medium is replaced with CCM with the addition of 5 μg/mL fibronectin and 5 μg/mL laminin, and the cells are cultured for 2 days.

At Stage II, the cells are switched into a medium prepared from 1:1 mixture of DMEM/F-12 medium containing 25 mM glucose, ITS-A, 0.45 mM IBMX, 5 μg/mL laminin and 5 μg/mL fibronectin for 1 day.

At Stage III, the cells are transferred into DMEM/F-12 medium containing 5.56 mM glucose, 10 mM nicotinamide, N2 supplement, B27 supplement, 5 μg/mL laminin and 5 μg/mL fibronectin for 4 days.

At Stage IV, the cells are transferred into a medium with the same supplements at the stage III of the Embodiment IV but containing 25 mM glucose for 3 days.

### Control Group I: Monolayer Culture

At Stage 0, undifferentiated human MSCs in monolayer are cultured in CCM for overnight.

At Stage I, the culture medium is replaced with CCM with the addition of 5 μg/mL fibronectin, and the cells are cultured for 2 days.

At Stage II, the cells are switched into a medium prepared from 1:1 mixture of DMEM/F-12 medium containing 25 mM glucose, ITS-A, 0.45 mM IBMX and 5 μg/mL fibronectin for 1 day.

At Stage III, the cells are transferred into DMEM/F-12 medium containing 5.56 mM glucose, 10 mM nicotinamide, N2 supplement, B27 supplement and 5 μg/mL fibronectin for 4 days.

At Stage IV, the cells are transferred into a medium with the same supplements at the stage III of the Control Group I but containing 25 mM glucose for 3 days.

### Control Group II: Monolayer Culture

At Stage 0, undifferentiated human MSCs in monolayer are cultured in CCM for overnight.

At Stage I, the culture medium is replaced with fresh CCM, and the cells are cultured for 2 days.

At Stage II, the cells are switched into a medium prepared from 1:1 mixture of DMEM/F-12 medium containing 25 mM glucose, ITS-A and 0.45 mM IBMX for 1 day.

At Stage III, the cells are transferred into DMEM/F-12 medium containing 5.56 mM glucose, 10 mM nicotinamide, N2 supplement and B27 supplement for 4 days.

At Stage IV, the cells are transferred into a medium with the same supplements at the stage III of the Control Group II but containing 25 mM glucose for 3 days.

Comparing the culture conditions of the above embodiments I-IV and control groups I and II, it is shown that the cells of the embodiments I-IV of the present invention are cultured for pellet suspension culture, and the cells of the control groups I and II are cultured for monolayer culture. The differences among the embodiments I-IV are the culture mediums with or without fibronection and/or laminin. In the embodiment I, the culture medium contains fibronectin; in the embodiment II, the culture medium contains neither fibronectin nor laminin; in the embodiment III, the culture medium contains laminin; in the embodiment IV, the culture medium contains both fibronectin and laminin. The differences between the control groups I and II are culture medium with or without fibronectin, wherein the culture medium in the control group I contains fibronectin, and that in the control group II does not.

The culture procedures of the control group II are the prior art for promoting differentiation of embryonic stem cells into IPCs. Firstly, in order to confirm whether the prior art promotes the differentiation of MSCs into IPCs or not, Immunofluorescence stains are performed for the cells in each stage of the control group II to detect several proteins, including insulin protein, an S-phase-associated nuclear antigen (Ki67) and neural markers that associated with neural precursor cells, such as Nestin and β-tubulin III. The percentage of positive-stained cells is calculated, so as to determine the expression level of each protein of the cells at each stage. The quantity of each marker of the cells at each stage is listed in Table 1.

TABLE 1
Percentage of positive cells, Mean (SD)

| Marker | Stage I | Stage II | Stage III | Stage IV |
|---|---|---|---|---|
| Ki67 | 61.8(5.7) | 45.3(2.5) | 24.7(3.4) | 13.3(2.3) |
| Nestin | 66.2(2.3) | 86.0(0.9) | 23.6(5.4) | 10.0(1.8) |
| β-tubulin III | 3.4(0.5) | 34.8(1.8) | 2.3(1.4) | 2.1(0.6) |
| Insulin | 2.3(0.5) | 1.3(0.3) | 2.0(0.9) | 3.6(0.6) |

It can be concluded from the results shown in Table 1 that the prior art, i.e. the method for promoting differentiation of embryonic stem cells into IPCs, can not promote the differentiation of MSCs into IPCs.

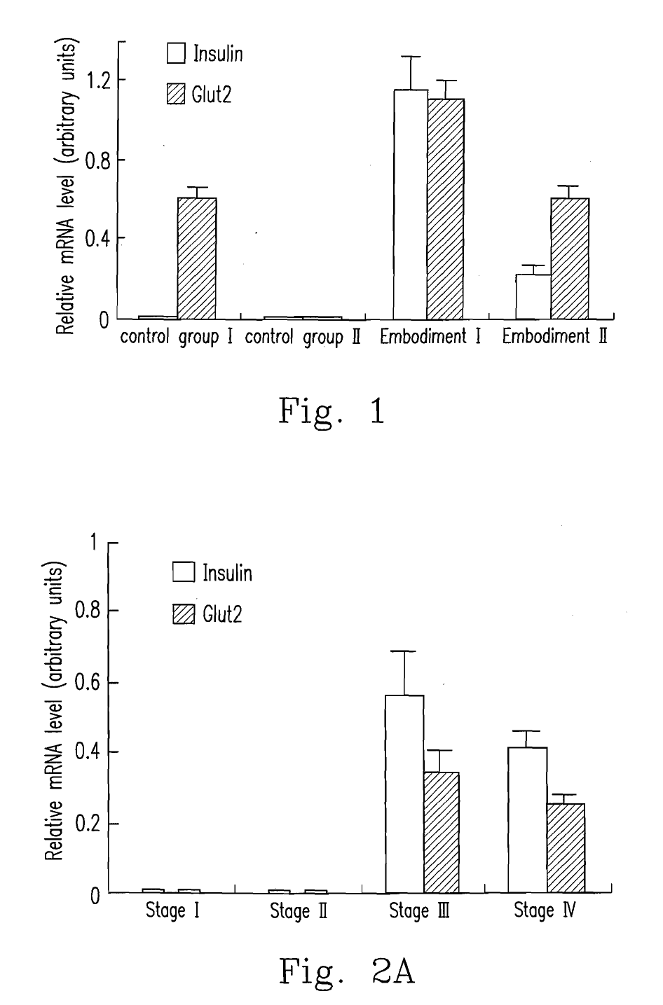

Islet cells are aggregated cells, and the extracellular matrix (ECM) thereof includes fibronectin and laminin. The present invention provides a method for promoting MSCs to differentiate into IPCs by cell pellet suspension culture with additional fibronectin or laminin. The cells of the embodiments and the control groups at the end of the four-stage culture are harvested, and the relative mRNA expression levels of insulin and glucose transporter 2 (Glut2) thereof are detected by reverse-transcription polymerase chain reaction (RT-PCR) and agarose electrophoresis, and are normalized by the expression level of β-actin. The measured results are shown in FIG. 1, and the primer sequences used in RT-PCR are shown in Table 2. In addition, it should be noted that all of the bar graphs shown in FIGS. 1-4 are represented by the measured mean with its standard deviation.

TABLE 2

| | |
|---|---|
| Gene | Primer sequence |

| | |
|---|---|
| β-Actin | 5′-GCACTCTTCCAGCCTTCCTTCC-3′ |
| | 5′-TCACCTTCACCGTTCAGTTTTT-3′ |
| Glut2 | 5′-AGGACTTCTGTGGACCTTATGTG-3′ |
| | 5′-GTTCATGTCAAAAAGCAGGG-3′ |
| Insulin | 5′-AACCAACACCTGTGCGGCTC-3′ |
| | 5′-AAGGGCTTTATTCCATCTCTCTCG-3′ |

As shown in FIG. 1, gene expressions of insulin are not detected in the control groups I and II, which the conventional monolayer culture method with and without fibronectin, respectively. On the other hand, gene expression of insulin is obviously detected in the present embodiment I, which is the pellet suspension culture method with fibronectin, and is slightly detected in the present embodiment II, which is the pellet suspension culture method without fibronectin. Glut2 expression is not detected control group II, moderately detected in the control group I and in the present embodiment II, and markedly detected in the present embodiment I.

Islet differentiation is further evaluated by dithizone (DTZ) staining to detect zinc ion, which binds six insulin molecules within the cells. Cells in the control groups I and II are not stained by DTZ; cells in the embodiment II are slightly stained by DTZ, and cells in the embodiment I are greatly stained by DTZ (not shown). Therefore, it can be concluded that the cell pellet suspension culture method provided in the present invention indeed promotes the MSCs to differentiate into IPCs, and the addition of fibronectin can further enhance the differentiation of MSCs into IPCs.

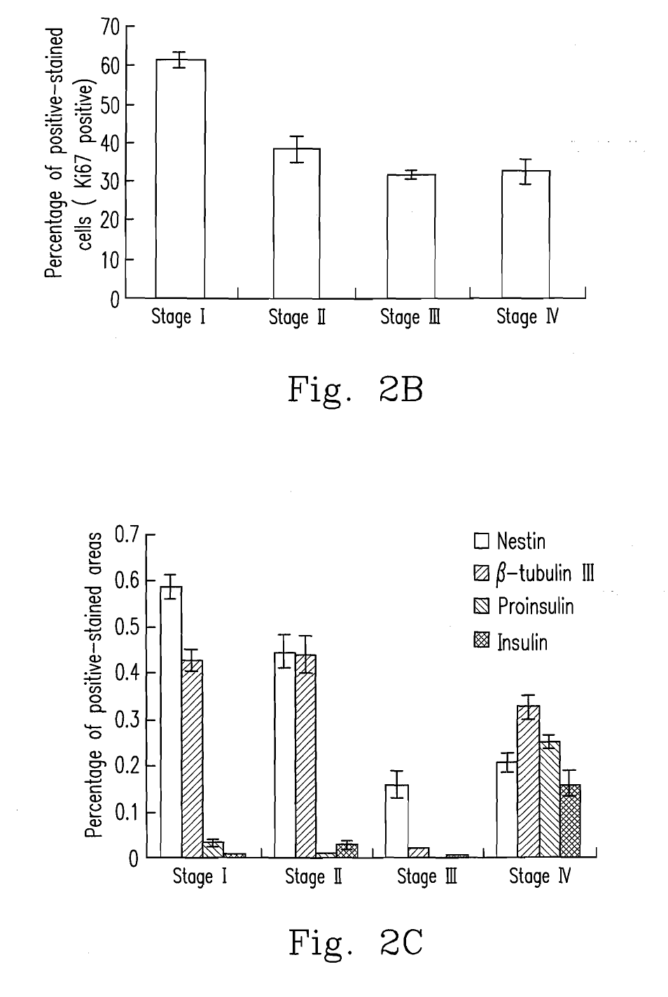

The gene expression profiles in all stages of the present embodiment I are analyzed by RT-PCR and agarose electrophoresis, and the detected results are further normalized by the expression level of β-actin. As shown in FIG. 2A, insulin and Glut2 are only detected in stage III and IV. In addition, immunohistochemistry is performed for detecting of neural markers and islet markers, including proinsulin and insulin, during stages I-IV. The percentage of positive-stained cells and areas are shown in FIGS. 2B and 2C. The protein, Ki67, which is related to mitosis, is mainly expressed in stage I and stage II cells. The protein, Nestin, which is related to neural precursor cells, is markedly expressed in stages I and II, as well. Insulin and proinsulin are mainly detected in stage IV cells, but not detected in cells before stage III.

Insulin is an endocrine for reducing blood sugar, and normal islet cells are induced by glucose to release insulin. In order to quantify insulin release by IPCs, a human insulin ELISA (Enzyme-Link ImmunoSorbent Assay) is employed herein.

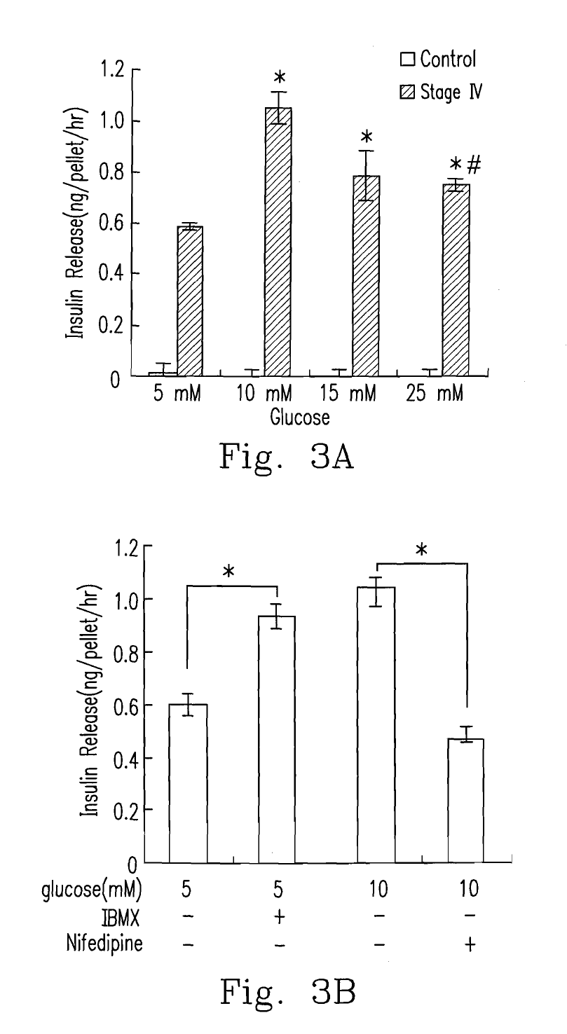

The suspension stage IV cells are rinsed twice in Phosphate Buffered Saline (PBS) and Krebs-Ringer bicarbonate (KRB) buffer (120 mM NaCl, 5 m M KCl, 2.5 m M CaCl₂, 1.1 mM MgCl₂, 25 mM NaHCO₃ , 0.1 BSA) and preincubated for 1 hour with KRB buffer containing 5 mM glucose. The suspension stage IV cells are then incubated for 1 hour in fresh KRB buffer with 5 mM, 10 mM, 15 mM or 25 mM glucose, and the released insulin in the incubation medium is quantified by ELISA. The quantified insulin release results are shown in FIG. 3A, wherein undifferentiated MSCs are served as a control (*p<0.05 as compared with 5 mM; #p<0.05 as compared with 10 mM; student's t test). As shown in FIG. 3A, the increase in glucose concentration to 10, 15 or 25 mM significantly increased the release of insulin by IPCs with the greatest release at 10 mM. The insulin secreted by IPCs derived from human MSCs in the present invention is up to 1.1 ng/h/2.5×10⁵ IPCs, which is even higher than those secreted by IPCs derived from rodent bone marrow stem cells in the prior art (referring to the citations 5-7).

Furthermore, in order to determine if the cell pellets use physiological signaling pathways to regulate insulin release, the effects of several agonists or antagonists, including IBMX (100 μM) and nifedipine (50 μM) are examined by respectively added in the fresh KRB buffer with 5 mM, 10 mM, 15 mM or 25 mM in the incubation step. Agonist-IBMX is an inhibitor of cyclic-AMP (cAMP) phosphodiesterase, and antagonist-nifedipine is a blocker of L-type Ca²⁺ channel (one of the Ca²⁺ channel present in β-cells). As shown in FIG. 3B, in the presence of low glucose concentration (5 mM), IBMX stimulates insulin release; conversely, in the presence of high glucose concentration (10 mM), nifedipine inhibits insulin release (*p<0.05, student's t test). Accordingly, it can be concluded that the IPCs derived MSCs in the present invention indeed regulate insulin release through physiological signaling pathway.

In order to further compare the different differentiation effects resulting from different ECMs when stem cells differentiate into IPCs in pellet suspension culture, the present invention performs the tests by adding fibronectin and laminin in combination or separately.

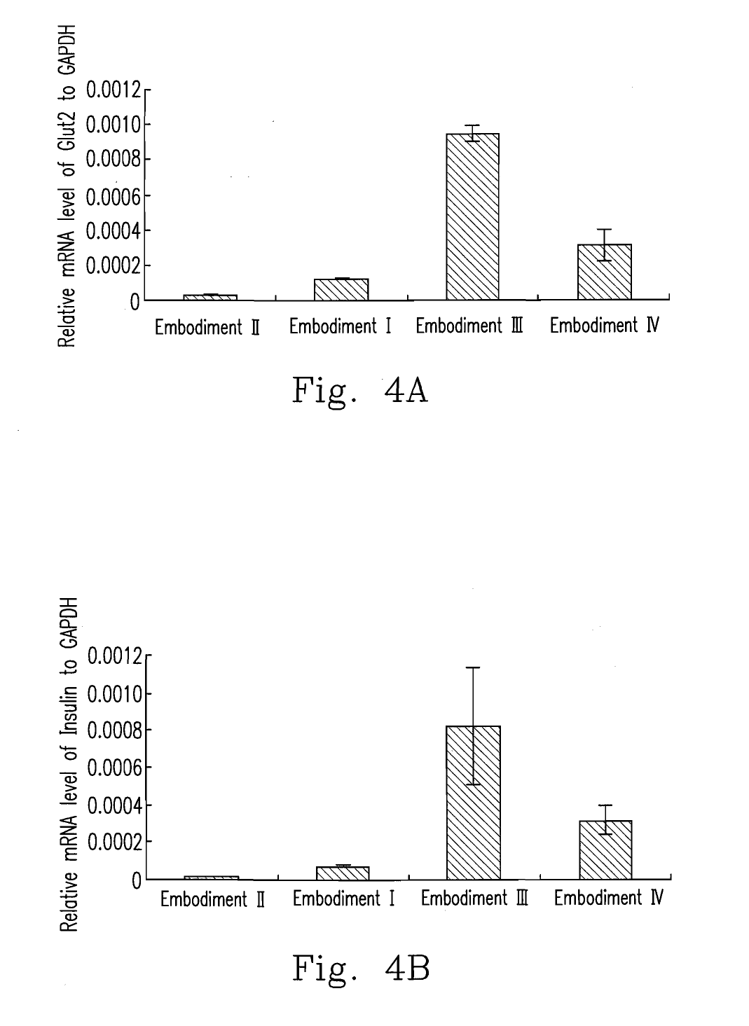

The gene expressions of insulin and Glut2 in stage IV cells of the present embodiments I-IV are analyzed by real-time RT-PCR respectively, and the detected results are further normalized by the expression level of glyceraldehyde-3-phosphate dehydrogenase (GAPDH) to measure the mRNA expression levels. The measured results are shown in FIGS. 4A and 4B. As shown in FIGS. 4A and 4B, the cells cultured by cell pellet suspension culture method with fibronectin and/or laminin have increased gene expressions of insulin and Glut2 by comparing with the cells cultured by cell pellet suspension culture method without any ECM. In addition, the embodiments III and IV, i.e. the cells cultured in the medium containing laminin and in the medium containing both fibronectin and laminin, have the insulin and Glut2 expressions higher than the embodiment I, i.e. the cells cultured in the medium containing only fibronectin. Moreover, it is found that the gene expression level of the embodiment III (containing laminin only) is significantly higher than that of the embodiment IV (containing both fibronectin and laminin).

It the previously studies, it is known that MSCs are plastic adherent and disperse without aggregation in culture. However, similar to islet cells, which are suspended cells and spontaneously form clusters after release from the pancreatic tissues, embryonic and neural stem cells are also suspended cells and aggregate to form clusters or spheres in the culture. Since MSCs have features different from those of embryonic stem cells, the prior art for promoting differentiation of embryonic stem cells into IPCs can not be applied to MSCs.

Based on the above, the present invention provides a different method for promoting the differentiation of stem cells into IPCs, wherein the MSCs are aggregated into a cell pellet to promote the differentiation, and it is preferable to add fibronection and/or laminin into the culture medium to enhance the differentiation. The IPCs derived from MSCs by the method provided by the present invention can release insulin higher than that of the IPCs derived from rodent bone marrow stem cells as illustrated in the prior art. Thus, it is apparent that the present invention has advantages over the prior art, hence it fits the demand of the industry and is industrially valuable.

Stem cells include embryonic stem cells and adult stem cells, wherein the generalized embryonic stem cells include embryonic germ cells, transformed embryonic stem cells and induced embryonic stem cells transformed from adult cells and a combination thereof. Adult stem cells include the stem cells obtained from different parts of human body, such as MSCs, haematopoietic stem cells, neural stem cells, etc., and MSCs can be derived from various organs, such as bone marrow, cord blood, adipose tissue and umbilical cord. Although the embodiments of the present invention only show the tested result by using MSCs, one skilled person in this field knows that most differentiation method can apply to most stem cells. Therefore, the present invention should be able to apply to embryonic stem cells, embryonic germ cells, transformed embryonic stem cells, induced embryonic stem cells, haematopoietic stem cells, neural stem cells or other adult stem cells and the combination thereof.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

### CITATIONS

1. Differentiation of embryonic stem cells to insulin-secreting structures similar to pancreatic islets. Science 2001; 292:1389-1394.
2. Differentiation of insulin producing cells from human neural progenitor cells. PLoS Med 2005; 2: e103.
3. Human bone marrow mesenchymal stem cells can express insulin and key transcription factors of the endocrine pancreas developmental pathway upon genetic and/or microenvironmental manipulation in vitro. Stem Cells 2005; 23: 594-603.
4. Differentiation of human embryonic stem cells into insulin producing clusters. Stem Cells 2004; 22: 265-274.
5. Little evidence of transdifferentiation of bone marrow-derived cells into pancreatic beta cells. Diabetologia 2003; 46: 1366-1374.
6. Adult bone marrow-derived cells trans-differentiating into insulin producing cells for the treatment of type I diabetes. Lab Invest 2004; 84: 607-617.
7. In vivo derivation of glucose-competent pancreatic endocrine cells from bone marrow without evidence of cell fusion. J Clin Invest 1996; 98: 2805-2812.

What is claimed is:

1. A method for promoting a differentiation of progenitor cells into insulin producing cells, comprising steps of:
suspending the progenitor cells in a first culture medium;
aggregating the progenitor cells to form a cell pellet; and
culturing the cell pellet in a second culture medium to promote the differentiation of the progenitor cells of the cell pellet into the insulin producing cells.

2. A method for promoting a differentiation of stem cells into insulin producing cells, comprising steps of:
suspending the stem cells in a first culture medium;
aggregating the stem cells to form a cell pellet; and
culturing the cell pellet in a second culture medium to promote the differentiation of the stem cells of the cell pellet into the insulin producing cells.

3. A method according to claim 2, wherein the stem cells are ones selected from a group consisting of embryonic stem cells, adult stem cells and a combination thereof.

4. A method according to claim 3, wherein the embryonic stem cells are ones selected from a group consisting of embryonic germ cells, transformed embryonic stem cells and induced embryonic stem cells transformed from adult cells and a combination thereof, and the adult stem cells are ones selected from a group consisting of mesenchymal stem cells, hematopoietic stem cells, neural stem cells and a combination thereof.

5. A method according to claim 4, wherein the mesenchymal stem cells are derived from a tissue being one selected from a group consisting of a bone marrow, a cord blood, an adipose tissue and an umbilical cord.

6. A method according to claim 2, wherein the cell pellet includes 2.5×10⁵ stem cells, and is suspended in the second culture medium.

7. A method according to claim 2, wherein the first culture medium is a complete culture medium including DMEM-low glucose, 10% fetal bovine serum, 100 U/mL penicillin and 10 μg/mL streptomycin.

8. A method according to claim 2, wherein the cell pellet is aggregated by centrifuging the stem cells at 200-600 g for 5-15 minutes.

9. A method according to claim 2, further comprising a step of preculturing the cell pellet in the first culture medium before the culturing step.

10. A method according to claim 2, wherein the second culture medium contains one selected from a group consisting of a fibronectin, a laminin and a combination thereof.

11. A method according to claim 2, wherein the culturing step includes further sub-steps of:
culturing the cell pellet for two days at a first stage;
culturing the cell pellet for one day at a second stage;
culturing the cell pellet for four days at a third stage; and
culturing the cell pellet for three days at a fourth stage.

12. A method according to claim 11, wherein:
the second culture medium is a complete culture medium at the first stage;
the second culture medium is a first DMEM/F-12 medium containing insulin-transferrin-selenium-A (ITS-A), 25 mM glucose and 0.45 mM 3-isobutyl-1-methylxanthine (IBMX) at the second stage;
the second culture medium is a second DMEM/F-12 medium containing N2 supplement, B27 supplement, 5.56 mM glucose and 10 mM nicotinamide at the third stage; and the second culture medium is a third DMEM/F-12 medium containing N2 supplement, B27 supplement, 25 mM glucose and 10 mM nicotinamide at the fourth stage.

13. A method according to claim 2, wherein the insulin producing cells are induced by a glucose to release an insulin, and the glucose has a concentration in a range of 5-25 mM.

14. A method according to claim 13, wherein the release of the insulin is enhanced by an inhibitor of cyclic-AMP phosphodiesterase.

15. A method according to claim 13, wherein the release of the insulin is inhibited by a blocker of Calcium ion channel.

16. An insulin producing cell differentiated from a stem cell, wherein the stem cell is aggregated into a cell pellet to be cultured so as to differentiate into the insulin producing cell.

17. An insulin producing cell according to claim 16, wherein the stem cell is one selected from a group consisting of an embryonic stem cell, an adult stem cell and a combination thereof.

18. An insulin producing cell according to claim 17, wherein the embryonic stem cell is one selected from a group consisting of an embryonic germ cell, a transformed embryonic stem cell and an induced embryonic stem cell transformed from an adult cell and a combination thereof, and the adult stem cell is one selected from a group consisting of a mesenchymal stem cell, a hematopoietic stem cell, a neural stem cell and a combination thereof.

19. An insulin producing cell according to claim 16, wherein the differentiation of the stem cell is promoted by one selected from a group consisting of a fibronectin, a laminin and a combination thereof.

20. An insulin producing cell according to claim 16, wherein the insulin producing cell is induced by a glucose to release an insulin, and the glucose has a concentration in a range of 5-25 mM.


SEQUENCE LISTING (6 sequences)

SEQ ID NO: 1 - DNA, 22 bp, ARTIFICIAL SEQUENCE
  Beta Actin primer 1
gcactcttccagccttccttcc

SEQ ID NO: 2 - DNA, 22 bp, ARTIFICIAL SEQUENCE
  Beta Actin primer 2
tcaccttcaccgttcagttttt

SEQ ID NO: 3 - DNA, 23 bp, ARTIFICIAL SEQUENCE
  Glut2 primer 1
aggacttctgtggaccttatgtg

SEQ ID NO: 4 - DNA, 20 bp, ARTIFICIAL SEQUENCE
  Glut2 primer 2
gttcatgtcaaaaagcaggg

SEQ ID NO: 5 - DNA, 20 bp, ARTIFICIAL SEQUENCE
  Insulin primer 1
aaccaacacctgtgcggctc

SEQ ID NO: 6 - DNA, 24 bp, ARTIFICIAL SEQUENCE
  Insulin primer 2
aagggctttattccatctctctcg